(19) United States
(12) Patent Application Publication
(10) Pub. No.: US 2020/0179507 A1
(43) Pub. Date: Jun. 11, 2020

(54) IMMORTALIZED CELL LINES AND METHODS OF USE

(71) Applicant: REGENTS OF THE UNIVERSITY OF MINNESOTA, MINNEAPOLIS, MN (US)

(72) Inventors: Douglas N. Foster, Hudson, WI (US); Linda K. Foster, Hudson, WI (US)

(21) Appl. No.: 16/461,222

(22) PCT Filed: Nov. 15, 2017
(86) PCT No.: PCT/US17/61804

§ 371 Date: May 15, 2019

Related U.S. Application Data
(60) Provisional application No. 62/423,439, filed on Nov. 17, 2016.

(51) Int. Cl. A61K 39/255; C12N 15/86; C12N 5/077

(52) U.S. Cl. CPC A61K 39/255; C12N 15/86; C12N 2720/12051; C12N 2510/04; C12N 2710/16651; C12N 5/0652

(57) ABSTRACT

Provided herein is a non-natural isolated chicken fibroblast cell that has the characteristics of immortalized growth and supporting replication of Marek's Disease Virus (MDV), wherein number of MDV produced by the cell is at least 1.5-fold greater than the number of the same MDV produced by a DF-1 cell under the same conditions. Also provided herein are methods including producing virus using the cell, determining the number of virus in a sample using the cell, and using the cell to produce protein.

### CROSS-REFERENCE TO RELATED APPLICATIONS

This application claims the benefit of U.S. Provisional Application Ser. No. 62/423,439, filed Nov. 17, 2016, which is incorporated by reference herein.

### SUMMARY OF THE APPLICATION

Provided herein is a non-natural isolated cell having the characteristics of immortalized growth and supporting replication of Marek's Disease Virus (MDV). The amount of MDV produced after 7 days of incubation in the cell is at least 1.5-fold greater than the amount of the same MDV produced by a DF-1 cell after 7 days under the same conditions. In one embodiment, the cell has the characteristics of LF-1 as deposited with the American Type Culture Collection on Sep. 17, 2016, in accordance with the provisions of the Budapest Treaty.

The isolated cell can include a virus, such as a herpesvirus (e.g., MDV), a retrovirus, or an influenza virus (e.g., an influenza virus of the genus Influenza A, Influenza B, or Influenza C). The isolated cell can include an exogenous recombinant vector, and the exogenous recombinant vector can include a retroviral vector, a coding region encoding a protein, or a combination thereof. Also provided is a subclone of the isolated cell, a culture that includes the isolated cell, and a composition that includes the isolated cell and a virus.

Also provided are methods for using the non-natural isolated cell. In one embodiment, a method includes incubating a composition including an isolated cell described herein and a virus under conditions suitable for replication of the virus in the isolated cell. The method can also include isolating virus produced during the incubating. In one embodiment, the method can also include passaging the virus on a culture of the isolated cell at least once and culturing the newly infected cells. The method can also include lysing cells that contain virus, preparing a seed virus from the virus produced during the incubating, producing a vaccine from the virus produced during the incubating, inactivating the virus produced during the incubating, or a combination thereof.

In one embodiment, a method is for determining the amount of virus in a sample. The method can include contacting a sample that includes virus with an isolated cell described herein to result in a mixture, incubating the mixture under conditions suitable for replication of the virus, identifying individual isolated cells infected with the virus, and determining the amount of virus in the sample. The method can further include preparing at least one serial dilution of a virus before the contacting.

In one embodiment, a method is for producing a protein. The method can include incubating an isolated cell described herein, wherein the isolated cell includes an exogenous coding region encoding a protein. In one embodiment, the exogenous coding region is part of a vector present in the isolated cell.

In one embodiment, a method is for growing cells. The method can include incubating a first cell with the isolated cell described herein under conditions suitable for replication of the first cell and the isolated cell.

In one embodiment, a method is for producing an immortalized cell. The method can include incubating DF-1 cells in culture, wherein the cells are plated at a low density to result in individual colonies after the incubating, isolating individual colonies of cells, testing cells of each colony for the characteristic of replicating Marek's Disease Virus at least 1.5-fold or at least 2-fold higher than replication of the Marek's Disease Virus in DF-1 cells under the same conditions

As used herein, “isolated” refers to material removed from its original environment (e.g., the natural environment if it is naturally occurring). An “isolated” cell is a cell that is no longer present in its natural environment.

The term “and/or” means one or all of the listed elements or a combination of any two or more of the listed elements.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure.

The terms “comprises” and variations thereof do not have a limiting meaning where these terms appear in the description and claims.

It is understood that wherever embodiments are described herein with the language “include,” “includes,” or “including,” and the like, otherwise analogous embodiments described in terms of “consisting of” and/or “consisting essentially of” are also provided.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

The above summary of the present disclosure is not intended to describe each disclosed embodiment or every implementation of the present disclosure. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### DETAILED DESCRIPTION OF ILLUSTRATIVE EMBODIMENTS

Provided herein is an isolated cell, and a culture of the cell (e.g., a population of the cells having a uniform genetic makeup, also referred to herein as a cell line). The cell described herein has been designated LF-1. LF-1 has the characteristic of being immortalized. As used herein, the terms “immortalized” and “immortalization” refer to non-rodent cells capable of growing in culture for greater than 30 passages and maintaining a population doubling level per day (pdl/day) of at least 0.5, or at least 0.6, and no greater than 0.9, or no greater than 0.8. In one embodiment, the population doubling level per day is 0.7. Population doubling level is the number of times cells in a population double since their primary isolation to determine when senescence occurs. Population doubling level is measured over a seven day period using 6 well plates to get the average in the linear range. Avian cells are generally considered immortalized after about 20 to about 25 passages in culture.

An LF-1 cell is not transformed. Immortalized cells are differentiated from transformed cells in that unlike transformed cells, immortalized cells are density dependent and/or growth arrested (e.g., contact inhibited). Transformed cells are capable of growth in soft agar and are usually able to form tumors when injected into laboratory animals. LF-1 is useful as a reservoir for growing virus or for expressing recombinant protein or virus, particularly where it is useful that the cells do not harbor contaminating virus or viral protein. The cells are also useful for studying the underlying mechanisms of cellular senescence and immortalization.

LF-1 is an avian cell and is derived from DF-1, a cell deposited with the American Type Culture Collection (ATCC) and designated UMNSAH-DF1 (see Foster et al., U.S. Pat. No. 5,672,485). Avian cells and human cells are known to be some of the most difficult cells to immortalize under tissue culture conditions. In avian fibroblasts, untreated cells typically last only 20-25 passages. That is, by 30 passages primary cultures of these avian cells are dead or dying. As described herein, the cell described herein was derived from DF-1 cells that had been passaged 69 times (see Example 1).

The DF-1 cell line used to produce the cell described herein was derived from chicken Embryo Fibroblastic (CEF) primary cells from 10 day old East Lansing Line (ELL-0) chicken embryos. The ELL-0 eggs and their layers were certified by the supplier as negative for Avian influenza (Type A), Avian reovirus, Avian adenoviruses (Groups Avian encephalomyelitis virus, Fowl pox, Newcastle disease virus, Paramyxovirus (Type 2), Mycoplasma, Salmonella and other infectious agents known to infect poultry stock.

An LF-1 cell has the characteristic of serving as a reservoir for growing the virus causing Marek's Disease, referred to herein as Marek's Disease Virus (MDV), and also referred to in the art as Gallid herpesvirus 2. Marek's Disease is a highly contagious viral disease in poultry, including chickens. LF-1 has the unexpected and advantageous characteristic of producing more MDV than DF-1. When LF-1 is used as the cell for replication of MDV, at least 1.5-fold, at least 2-fold, or at least 2.5-fold more MDV is produced compared to the production of MDV by DF-1 under the same conditions. In one embodiment, no more than 3-fold more MDV is produced compared to the production of MDV by DF-1 under the same conditions. In one embodiment, to determine production of MDV, LF-1 cells and DF-1 cells can be seeded in multiwell plates at 1×10⁶ cells per 6 cm well and grown at 41° C. for 24 hours. The medium can be Dulbecco's Modified Eagle Medium (DMEM) containing 4.5 g/L glucose, 10% Fetal Bovine Serum, 4 mM L-glutamine; 100 units/ml penicillin-100 ug/ml streptomycin solution. At 24 hours cell media can be changed and replaced with media containing 2% to 5% Fetal Bovine Serum and Dulbecco's Modified Eagle Medium (DMEM) containing 1.0 g/L glucose, and the cells are infected with Marek's Disease Virus. In one embodiment, a Marek's Disease Virus useful in determining whether a cell has the characteristic of producing more MDV than DF-1 cells includes a virulent MDV. A MDV is considered virulent if it can cause in a chicken one or more symptoms of classical Marek's disease (neurolymphomatosis), acute Marek's disease, ocular lymphomatosis, cutaneous Marek's disease, atherosclerosis, or immunosuppression. In one embodiment, a Marek's Disease Virus that is not useful in determining whether a cell has the characteristic of producing more MDV than DF-1 cells includes an attenuated MDV, e.g., a MDV that causes little if any of the symptoms of MDV infection in a chicken. An example of a suitable MDV is T-HVT-1 (Ft. Dodge Animal Health, Ft. Dodge, Iowa). Stock virus can be added as a 1:100 or 1:250 dilution at an approximate multiplicity of infection (m.o.i.) of 0.0025 and 0.001, respectively. Following 7 days of incubation at 41° C. the wells can be analyzed to determine the amount of virus produced. Other methods can be used to determine the production of MDV by LF-1 cells and DF-1 cells. An example of a suitable assay for measuring virus is the use of anti-MDV antibody to detect plaques (see Example 2).

The LF-1 cell has been deposited with American Type Culture Collection (ATCC), 10801 University Boulevard, Manassas, Va., 20110-2209, USA, on Sep. 17, 2016, and has the ATCC Patent Deposit Designation PTA-123527. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. § 112.

DF-1 cells are able to support the replication of Infectious Bursal Disease Virus (Rekha K., et al. 2014, Bio Med Res. Internat #494835), Avian Influenza (Lee, C-W. et al., 2008, J Virol. Meth. 153, 22-28), Newcastle Disease Virus (DiNapoli, et al., 2007, J. Virol. 81 (21) 11560-11568, and Nayak, B., et al., 2009, PLOS One, 4(8):e6509, doi 10.1371), Avian Sarcoma and Leukosis Virus Subgroup A (Chen W. et al., 2015, Sci. Report 5, 9900 doi 10. 1058), Avian retroviruses (Birmingham C. L. et al., 2013, J. Clin. Microb. 51 (5) 1496-1504), and Modified Vaccinia Ankara (MVA) (Alberca B. et al., Vaccine 32 (29), 3670-3674, 2014), HVT (Herpesvirus of Turkeys), avian herpesvirus (serotype III), Fowl Pox virus, reovirus, and Rous Sarcoma Virus, and it is expected that LF-1 can also serve as a substrate for production of these viruses. LF-1 can be tested for its ability to replicate Circodnavirideae, chicken HSV serotype II and other viruses, including non-avian viruses. For instance, it is expected that LF-1 will support the replication of influenza virus (including genus Influenza A, Influenza B, and Influenza C), Infectious Bursal Disease Virus, Tick-Borne Encephalitis Virus, and viruses causing measles, mumps, rubella, smallpox, rabies, and yellow fever. In one embodiment, LF-1 is useful for retrovirus production, because the parental DF-1 cell and the layers from which DF-1 was ultimately derived did not have detectable retrovirus infection. Virus produced by LF-1 can be wild-type, and optionally virulent, or can be virus that has been modified, including attenuated. Virus can be obtained from a variety of sources. For instance, virus can be obtained from clinical samples of a subject known to be or suspected of being infected with the virus, or virus that is readily available from commercial sources can be used. Virus produced as described herein can be useful for the manufacture of vaccine, such as an attenuated virus.

To produce virus, LF-1 can be seeded into tissue culture flasks, roller bottles, stir culture, into hollow fiber reactors or other mass culture systems such as high density bioreactors (M. Mel, et al., Med. J. Malaysia, 65: 19-21, 2010). For roller bottle virus propagation, the cells are seeded at about 2-5×10⁴ cells/cm² of surface area. The multiplicity of infection (ratio of infectious virus particles to cells) to initiate virus growth will vary depending on virus strain. Those skilled in the art of virology and skilled in the growth of particular viruses and strains of viruses will be able to maximize virus yield through the standard manipulation of the multiplicity of infection, temperature, media variations, and the like, without undue experimentation.

Methods for harvesting the virus after infection of LF-1 to obtain infectious virus stock also varies with virus strain. Enveloped viruses egress into the culture media more slowly than non-enveloped virus. Stocks of virus can be obtained from the culture media alone or from cell lysates pooled with the conditioned media. For lytic viruses (those efficient at lysing a cell during virus egress), harvesting the conditioned culture media (e.g., spent media containing virus) after a gentle centrifugation step to remove cell debris can be sufficient. Methods for harvesting and saving virus from a wide range of virus strains are known in the art. A virus stock can be used in preparing a seed virus that can be used in vaccine manufacture.

There are a variety of methods, also all known in the art, for measuring virus growth from a culture of cells. For example, the titer of a virus stock for members of the Herpesvirus family and for a variety of viruses producing foci of cytopathology on a cell monolayer surface are readily measured by plaque assay (as plaque forming units/ml of culture fluid or as plaque forming units/dose for vaccine inoculum virus quantitation) or as tissue culture infectious dose-50 (TCID₅₀). Rapidly lytic viruses are often better measured by TCID₅₀ as the dose or dilution of virus stock capable of infecting 50% of the cultures in a defined time period. Methods for growing and quantitating virus are known in the art and sources for teaching virus quantification methods can be found in, for instance, Fields, et al. (eds) Fundamental Virology 1991, Raven Press, New York or in Mandell, et al. (eds.) Principles and Practice of Infectious Diseases, 1985, John Wiley & Sons, New York.

Virus produced using the cell disclosed herein can be used in the manufacture of vaccine. For instance, virus can be attenuated using methods that are routine and known in the art. Virus can be inactivated, for instance killed, using methods that are routine and known in the art. Virus produced using the cell disclosed herein can be prepared in a composition that is suitable for pharmaceutical administration.

In addition to supporting virus growth, LF-1 can be used as a packaging line to produce recombinant virus, including retrovirus. LF-1 can also be used to produce recombinant proteins, including viral proteins, and the like. Methods for incorporating nucleic acid that encodes a protein into a vector under the control of regulatory elements capable of directing expression of a protein in a eukaryotic cell, such as LF-1, are known in the art. Expression vectors are replicable nucleic acid fragments that can direct expression of a recombinant protein. Replicable expression vector components generally include, but are not limited to, one or more of the following: an origin of replication, one or more marker genes, enhancer elements, promoter elements, optional signal sequences and transcription termination sequences. The selection or marker genes encode protein that serves to identify a population of transformed or transfected cells. Typical selection genes encode proteins that confer resistance to antibiotics or other toxins, complement auxotrophic deficiencies, or supply critical nutrients not available from complex media.

Expression vectors having nucleic acid encoding recombinant protein can be transfected into LF-1 and can be used to direct expression of the recombinant protein in the cell. The vector can encode any recombinant protein capable of expression in chicken embryonic fibroblast cells, including, but not limited to, virus protein, including reverse transcriptase and/or viral structural protein. Examples of vectors to produce recombinant protein in a cell include retroviral vectors to produce tumor suppressive protein, or viral structural protein such as those disclosed by Givol, et al. Oncogene 11(12):2609-2618, 1995, Givol, et al. Cell Growth & Differentiation 5(4):419-429, 1994, Akiyama, et al. Virology 203(2):211-220, 1994 and Boyer, et al. Oncogene 20:457-66, 1993.

LF-1 can serve as substrate to express recombinant virus, including, but not limited to, recombinant retrovirus. LF-1 is suitable to serve as a packaging cell line for genetically engineered virus useful for gene therapy, or the like. Constructs and methods for using a particular cell line as a packaging cell line are known in the art. For example, Boerkoel, et al. (Virology 195(2):669-79, 1993) discloses methods for packaging virus using primary chicken embryonic fibroblasts as the packaging cell line. These same methods can be used to package virus in LF-1.

Since most avian cell lines and all transformed avian cells as well as virtually all mouse transformed cell lines either contain viral contaminants such as endogenous virus or produce viral protein, they are not desirable for the production of vaccines for use in humans and animals. The cells cannot be used to produce recombinant protein because the endogenous contaminants can contaminate purified recombinant protein preparations. Advantageously, LF-1 provide a suitable alternative to these problems.

LF-1 can also serve as a substrate for supporting virus growth from other cells. These other cells include primary cells, or cultured cells that show improved growth or longevity in culture in the presence of other cells, or in the presence of extracellular matrix proteins such as collagens, laminins, and the like. In one embodiment, cells are mixed with virus and then mixed with LF-1 cells. The ratio of LF-1 cells to another type of cell can be, for instance, 5:1 to 20:1, such as 10:1 (10 LF-1 cells to 1 other type of cell). The mixed cells are then placed into culture. In a second embodiment the cells are mixed with virus and plated onto the surface of LF-1 cells already attached to a tissue culture surface. LF-1 serve as a support for the other cells and, without intending to limit the scope of this disclosure, LF-1 can supply growth factors and the like as well as extracellular matrix components, and the like to support the other cells while they are producing virus.

### EXEMPLARY EMBODIMENTS

### Embodiment 1

A non-natural isolated chicken fibroblast cell comprising the characteristics of immortalized growth and supporting replication of Marek's Disease Virus (MDV), wherein number of MDV produced by the cell is at least 1.5-fold greater than the number of the same MDV produced by a DF-1 cell under the same conditions.

### Embodiment 2

The isolated cell of embodiment 1 wherein the conditions comprise infecting cells with a test MDV at a multiplicity of infection of 0.0025 to 0.001 followed by incubation of the cells for 7 days at 41° C. in Dulbecco's Modified Eagle Medium containing 4.5 g/L glucose and 10% Fetal Bovine Serum and 4 mM L-glutamine, and detecting plaques, wherein the number of plaques is indicative of the number of MDV produced.

### Embodiment 3

The isolated cell of any one of embodiments 1-2 wherein the isolated cell comprises a virus.

### Embodiment 4

The isolated cell of embodiment 3 wherein the virus is a herpesvirus.

### Embodiment 5

The isolated cell of embodiment 4 wherein the virus is MDV.

### Embodiment 6

The isolated cell of embodiment 3 wherein the virus is a retrovirus.

### Embodiment 7

The isolated cell of embodiment 3 wherein the virus is an influenza virus.

### Embodiment 8

The isolated cell of embodiment 7 wherein the influenza virus is selected from the genus Influenza A, Influenza B, and Influenza C.

### Embodiment 9

The isolated cell of any one of embodiments 1-8 wherein the isolated cell comprises an exogenous recombinant vector.

### Embodiment 10

The isolated cell of embodiment 9 wherein the exogenous recombinant vector comprises a retroviral vector.

### Embodiment 11

The isolated cell of embodiment 9 wherein the exogenous vector comprises a coding region encoding a protein.

### Embodiment 12

The isolated cell of any one of embodiments 1-11 wherein the test MDV is a virulent MDV.

### Embodiment 13

The isolated cell of any one of embodiments 1-12 wherein the test MDV is not an attenuated MDV.

### Embodiment 14

The cell of any one of embodiments 1-13 wherein the cell has the characteristics of LF-1 as deposited with the American Type Culture Collection under number PTA-123527 in accordance with the provisions of the Budapest Treaty.

### Embodiment 15

A subclone of the isolated cell of any one of embodiments 1-14.

### Embodiment 16

A culture comprising the isolated cell of any one of embodiments 1-15.

### Embodiment 17

A composition comprising the isolated cell of any one of embodiments 1-16 and a virus.

### Embodiment 18

A method for producing virus comprising:

incubating a composition comprising the isolated cell of any one of embodiments 1-17 and a virus under conditions suitable for replication of the virus in the isolated cell.

### Embodiment 19

The method of embodiment 18 further comprising isolating virus produced during the incubating.

### Embodiment 20

The method of any one of embodiments 18-19 further comprising passaging the virus on a culture of the isolated cell at least once, and culturing the newly infected cells.

### Embodiment 21

The method of any one of embodiments 18-20 wherein the isolating comprises lysing cells that contain virus.

### Embodiment 22

The method of any one of embodiments 18-21 further comprising preparing a seed virus from the virus produced during the incubating.

### Embodiment 23

The method of any one of embodiments 18-22 further comprising producing a vaccine from the virus produced during the incubating.

### Embodiment 24

The method of any one of embodiments 18-23 further comprising inactivating the virus produced during the incubating.

### Embodiment 25

The method of any one of embodiments 18-24 wherein the virus is a herpesvirus.

### Embodiment 26

The method of any one of embodiments 25 wherein the virus is MDV.

### Embodiment 27

The method of any one of embodiments 18-24 wherein the virus is a retrovirus.

### Embodiment 28

The method of any one of embodiments 18-24 wherein the virus is an influenza virus.

### Embodiment 29

The method of embodiment 28 wherein the influenza virus is selected from the genus Influenza A, Influenza B, and Influenza C.

### Embodiment 30

A method for determining the number of virus in a sample comprising:

contacting a sample comprising virus with the isolated cell of any one of embodiment 1-16 to result in a mixture;

incubating the mixture under conditions suitable for replication of the virus;

identifying individual isolated cells infected with the virus; and

determining the number of virus in the sample.

### Embodiment 31

The method of embodiment 30 further comprising preparing at least one serial dilution of a virus before the contacting.

### Embodiment 32

A method for producing a protein, comprising:

incubating the isolated cell of any one of embodiment 1-16, wherein the isolated cell comprises an exogenous coding region encoding a protein.

### Embodiment 33

The method of embodiment 32 wherein the exogenous coding region is part of a vector present in the isolated cell.

### Embodiment 34

A method for growing cells comprising:

incubating a first cell with the isolated cell of any one of embodiment 1-16 under conditions suitable for replication of the first cell and the isolated cell.

### Embodiment 35

A method for producing an immortalized cell comprising:

incubating DF-1 cells in culture, wherein the cells are plated at a low density to result in individual colonies after the incubating;

isolating individual colonies of cells;

testing cells of each colony for the characteristic of replicating Marek's Disease Virus at least 2-fold higher than replication of the Marek's Disease Virus in DF-1 cells under the same conditions.

### Embodiment 36

The method of embodiment 35 wherein the conditions comprise DMEM, 1.0 grams/liter glucose and 2% fetal bovine serum.

The present disclosure is illustrated by the following examples. It is to be understood that the particular examples, materials, amounts, and procedures are to be interpreted broadly in accordance with the scope and spirit of the disclosure as set forth herein.

### Example 1

### Obtaining the LF-1 Clonal Variant Cell Line

The morphology of DF-1 cells from passages 69 (P69) and 174 (P174) was compared to determine if any changes had occurred over the span of >100 passages in culture. In order to visualize the morphology, limiting dilutions of cells were made. DF-1 P69 cells were seeded very sparsely (2×10³ cells) onto a 100 mm dish. Cloning rings were used to pick 6 different clones of cells that were presumably derived from a single cell and were thus considered to be a pure clonal foci of cells. The cell clones were subsequently removed and individually seeded in a 6 well plate and allowed to grow. After approximately a month, clones 601 through 606 had proliferated enough in order to be split and were placed in 100 mm dishes. Eleven days later clones 601 through 605 were frozen in liquid nitrogen (clone 606 grew inordinately fast and was not selected for freezing). Clones 601, 604 and 605 all had growth rates of 0.7 pdl/day (similar to DF-1 P69) and were negative for tumorigenic growth as analyzed by soft agar assay. The morphology of clones 601 to 605 were uniformly fibroblastic in nature and essentially indistinguishable from most of the parental DF-1 P69 cells. DF-1 clone 604 was subsequently renamed as the LF-1 cell line.

### Example 2

### Analysis of Marek's Disease Virus Propagation

The clonally selected LF-1 variant of DF-1 was tested for Marek's Disease Virus to determine if the LF-1 pure cell population was any different than the population of the parental DF-1 cell line. The LF-1 cell line was compared to DF-1 P261 cells (where P261 refers to passage 261). Chicken embryo primary skin cells and the QT-35 quail cell line served as positive controls. Cells were seeded at 1×10⁶ cells per 6 cm well in multiwall plates. After 24 hours cell media was changed and replaced with media containing 5% FBS and DMEM containing 1.0 g/L glucose. Cells were infected with Marek's Disease Virus (T-HVT-1, from Ft. Dodge Animal Health, Ft. Dodge, Iowa). Stock virus was added as a 1:100 or 1:250 dilution at an approximate multiplicity of infection (m.o.i.) of 0.0025 and 0.001, respectively. Following 7 days of incubation at 41° C. cells were prepared for IFA staining by removing the medium and any virus present in the medium, washing with PBS, fixing with 95% EtOH, and incubating with a primary antibody from Merek's Disease Virus infected SPAFAS Chicken Embryo Fibroblast primary cells, followed by a secondary antibody (FITC-conjugated rabbit anti-chicken IgG, SAB 3700225, Sigma, St. Louis, Mo.). Immunofluoresence was read using a Nikon inverted fluorescent microscope. Titers were calculated as: [plaque forming units (pfu)]×[the dilution factor of the virus stock added]×[0.2 ml volume (the amount that would be given to a bird in field trial testing]. The results shown in Table 1 are the average of duplicate assays.

TABLE 1

| Cell type: | LF-1 | DF-1 P261 | CEF primary skin | QT35 |
|---|---|---|---|---|
| Titer: | 2100 | 975 | 2760 | 1400 |

The LF-1 cells demonstrated a 2.15 fold increased HVT titer compared to the DF-1 P261 cells.

This experiment was repeated as described above with one change: the serum concentration was reduced to 2% FBS during HTV infection. The cells were infected for 6 days. The results shown in Table 2 are the average of duplicate assays.

TABLE 2

| Cell type: | LF-1 | DF-1 P261 | CEF primary skin | QT35 |
|---|---|---|---|---|
| Titer: | 2175 | 810 | 3096 | 60 |

The LF-1 cells demonstrated a 2.69 fold increased HVT titer compared to the DF-1 P261 cells.

### Example 3

### Growth and Storage Conditions for LF-1 Cells

LF-1 cells were grown using the same conditions of the parental DF-1 cells: Invitrogen (Gibco) DMEM with 4.5 g/L glucose; 4 mM L-glutamine; 10% FBS; 1% antibiotic/mycotic solution (50 units penicillin and 50 ug streptomycin/ml of growth media).

LF-1 cells were frozen using the same conditions of the parental DF-1 cells: Invitrogen (Gibco) DMEM with 4.5 g/L glucose; 4 mM L-glutamine; 20% FBS; 12% DMSO

### Example 4

### Further Characterization of LF-1 Cells

The LF-1 cell line was tested for potential mycoplasma contamination using a commercial kit (Stratagene, Inc. San Diego Calif.) and was found to be negative.

Soft agar analysis for determining contact inhibition status of the LF-1 cell line was measured as described in Foster et al. (U.S. Pat. No. 5,672,485). The LF-1 cell line was negative, indicating the LF-1 cell line does not have tumorigenic potential.

Reverse transcriptase activity was not analyzed but has been repeatedly assayed for in the parental DF-1 cell line (see Foster et al., U.S. Pat. No. 5,672,485, Example 2). The parental DF-1 cell line is negative, and it is expected that the LF-1 cell line is negative for reverse transcriptase activity. The absence of reverse transcriptase activity suggests the LF-1 cell line is free of virus that expresses reverse transcriptase. Evaluation of reverse transcriptase activity in LF-1 can be determined as described at Example 2 of Foster et al. (U.S. Pat. No. 5,672,485).

Tumorgenicity of the LF-1 cell line by injection of the cells into test animals was not analyzed. However, this has been assayed for in the parental DF-1 cell line (see Foster et al., U.S. Pat. No. 5,672,485). The injection of actively growing parental DF-1 cells into adult chickens did not result in observable tumors, and it is expected that the LF-1 cell line is also nontumorigenic.

The complete disclosure of all patents, patent applications, and publications, and electronically available material cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The disclosure is not limited to the exact details shown and described, for variations obvious to one skilled in the art will be included within the disclosure defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present disclosure. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A non-natural isolated chicken fibroblast cell comprising the characteristics of immortalized growth and supporting replication of Marek's Disease Virus (MDV), wherein number of MDV produced by the cell is at least 1.5-fold greater than the number of the same MDV produced by a DF-1 cell under the same conditions.

2. The isolated cell of claim 1 wherein the conditions comprise infecting cells with a test MDV at a multiplicity of infection of 0.0025 to 0.001 followed by incubation of the cells for 7 days at 41° C. in Dulbecco's Modified Eagle Medium containing 4.5 g/L glucose and 10% Fetal Bovine Serum and 4 mM L-glutamine, and detecting plaques, wherein the number of plaques is indicative of the number of MDV produced.

3. The isolated cell of claim 1 wherein the isolated cell comprises a virus.

4. (canceled)

5. The isolated cell of claim 4 wherein the virus is MDV.

6. (canceled)

7. (canceled)

8. (canceled)

9. The isolated cell of claim 1 wherein the isolated cell comprises an exogenous recombinant vector.

10. The isolated cell of claim 9 wherein the exogenous recombinant vector comprises a retroviral vector.

11. The isolated cell of claim 9 wherein the exogenous vector comprises a coding region encoding a protein.

12. (canceled)

13. (canceled)

14. The cell of claim 1 wherein the cell has the characteristics of LF-1 as deposited with the American Type Culture Collection under number PTA-123527 in accordance with the provisions of the Budapest Treaty.

15. A subclone of the isolated cell of claim 1.

16. A culture comprising the isolated cell of claim 1.

17. A composition comprising the isolated cell of claim 1 and a virus.

18. A method for producing virus comprising:
incubating a composition comprising the isolated cell of claim 1 and a virus under conditions suitable for replication of the virus in the isolated cell.

19. The method of claim 18 further comprising isolating virus produced during the incubating.

20. The method of claim 18 further comprising passaging the virus on a culture of the isolated cell at least once, and culturing the newly infected cells.

21. The method of claim 19 wherein the isolating comprises lysing cells that contain virus.

22. (canceled)

23. The method of claim 1 further comprising producing a vaccine from the virus produced during the incubating.

24. The method of claim 1 further comprising inactivating the virus produced during the incubating.

25. (canceled)

26. (canceled)

27. (canceled)

28. (canceled)

29. (canceled)

30. A method for determining the number of virus in a sample comprising:
contacting a sample comprising virus with the isolated cell of claim 1 to result in a mixture;
incubating the mixture under conditions suitable for replication of the virus;
identifying individual isolated cells infected with the virus; and
determining the number of virus in the sample.

31. (canceled)

32. A method for producing a protein, comprising:
incubating the isolated cell of claim 1, wherein the isolated cell comprises an exogenous coding region encoding a protein.

33. (canceled)

34. A method for growing cells comprising:
incubating a first cell with the isolated cell of claim 1 under conditions suitable for replication of the first cell and the isolated cell.

35. (canceled)

36. (canceled)
